(19) United States
(12) Patent Application Publication
(10) Pub. No.: US 2012/0115828 A1
(43) Pub. Date: May 10, 2012

(54) MEDICINAL CREAM CONTAINING MICONAZOLE NITRATE, HYDROCORTISONE ACETATE, AND A BIOPOLYMER, AND A PROCESS TO MAKE IT

(75) Inventors: Vanagamudi Subramaniam Sulur, Chennai (IN); Madhavan Srinivasan, Chennai (IN); Neelakandan Narayanan Chulliel, Chennai (IN); Balkrishnana Selvaraj, Chennai (IN)

(73) Assignee: APEX LABORATORIES PRIVATE LIMITED, CHENNAI, TN (IN)

(21) Appl. No.: 13/264,995

(22) PCT Filed: Apr. 14, 2010
(86) PCT No.: PCT/IB10/51610

§ 371 Date: Jan. 3, 2012

(30) Foreign Application Priority Data
Apr. 20, 2009 (IN) 1020/MUM/2009

(51) Int. Cl. A61K 31/573; A61P 29/00; A61P 17/02; A61P 31/10

(52) U.S. Cl. 514/171

(57) ABSTRACT

The present invention is directed to a composition for treating bacterial skin infections & skin inflammation, along with skin rejuvenation. More particularly, the present invention relates to a pharmaceutical cream comprising a biopolymer, a corticosteroid and an antibacterial active ingredient. It discloses a composition for treating fungal skin infections & skin inflammation, along with skin rejuvenation containing a) a biopolymer in the form of chitosan, b) a combination of active pharmaceutical ingredients (APIs), miconazole and hydrocortisone acetate used in treating bacterial skin infections & skin inflammations, c) a cream base containing primary and secondary emulsifiers, waxy materials, co-solvents, acids, preservatives, buffering agents, anti oxidants, chelating agents, and humectants and d) water. The active ingredients, namely chitosan, corticosteroid in the form of hydrocortisone acetate, and an antibacterial agent in the form of miconazole, are incorporated in cream base for use in treating bacterial skin infections and skin inflammation due to allergy & itching, & wounds on human skin involving contacting human skin with the above identified composition.

### FIELD OF INVENTION

The present invention relates to a composition for treating fungal skin infections & skin inflammation, along with skin rejuvenation. More particularly, the present invention relates to a pharmaceutical cream comprising a biopolymer in the form of chitosan, a corticosteroid in the form of Hydrocortisone Acetate and an antifungal active ingredient in the form of Miconazole Nitrate.

### BACKGROUND OF THE INVENTION

Skin disorders can be broadly categorized as those arising from bacterial forms or fungi. Antifungal or antibacterial compositions are traditionally applied as lotions, creams or ointments. Furthermore in many instances, it is difficult to ascertain whether the skin condition is due to a bacterial agent or a fungus.

One approach to treating skin disorders is through elimination by trial and error. Antibacterial or antifungal compositions are applied in turn and response monitored and treatment modified. A major disadvantage of this approach is that treatment needs to be applied many times a day during the treatment period. This is greatly inconvenient and also not cost effective for a majority of human population, particularly in the under-developed nations.

There are several treatments available to treat skin disorders caused by bacteria or fungi. Typically, such compositions use steroids, antibacterial agents or antifungal agents, (or a fixed dose combination of these) and focus on these pharmaceutically active ingredients. The composition of such formulations is such as to enhance their physical/chemical/bio-release profile.

Many skin disorders caused by inflammation and bacterial attacks lead to itching and subsequent scratching, which, among other causes, can in turn lead to serious and complicated secondary infections. The conventionally available treatments do not focus on skin healing or rejuvenation; normally these two aspects are left to heal naturally.

The word healing as related to compromised skin conditions (cuts, wounds, infections, inflammations, abrasions, etc.) are not only about prevention, control, elimination of the source cause such as bacteria or fungi but also to restore the skin to its pre-infection state.

The current approaches of skin treatment can be broadly categorized into two stages, a. healing b. restoration of skin to pre-ailment state. The healing part comprises elimination, to the best possible extent, of the root cause of the disorder. This may be elimination of bacteria or fungi causing the infection through a suitable treatment of antibacterial or antifungal agents or reducing the inflammation through steroid treatment. While this treatment is under way, the ongoing compromised condition of the skin continues to be susceptible to secondary infections which can be of quite serious nature. In the case of scratched or wounded skin, it is important for blood clotting to occur quickly as it reduces chances of secondary infections. The focus of such treatments, which are administered through creams, lotions, ointments is on the action of active pharmaceutical ingredients. Cream bases or ointment bases are merely viewed as carriers to take APIs to the sites of disorder.

However, the aspect of restoring the skin back to its pre-disorder state is almost completely left to nature. Therefore one key drawback of the existing skin treatment approaches is that they run the risk of secondary infections due to slow blood clotting and wound healing process.

Furthermore, from the study of the prior art several lacking aspects of the existing prescription derma products used for topical treatment of skin disorders. This is manifested by the fact that the cream base matrix or the ointment base has been overlooked for any potential therapeutic benefits. In particular none of the available prior art suggests that:
Topical skin formulations can deliver skin healing or regeneration beyond the activity of the main APIs such that the therapeutic outcome of the main APIs is enhanced.
The addition of biologically active polymers (the so-called biopolymers) is a complex process in which the stability of the formulations could be compromised if the right biopolymer or naturally interacting formulation excipients or process parameters are not well thought through and optimized to enhance and complement therapy outcomes at the drug design stage itself.
Incorporation of a functionally bio-active excipient polymer in cream matrix while retaining the functional stability of the API in a single dose format of dermaceutical cream involves resolution of problems specific to the physical stability of cream matrix.

A look at some of the existing patents illustrates the above points.

CN 1931164 deals with the nanometer miconazole nitrate emulsion medicine which consists of surfactant, oil, miconazole nitrate and distilled water. The application claims novelty on the assertion that the nanometer miconazole nitrate emulsion has high skin permeability, no contamination to clothing, high dissolubility of miconazole nitrate, raised bioavailability of miconazole nitrate, delayed metabolism time and wide medicine market foreground.

U.S. Pat. No. 5,461,068 pertains to improved formulations for topical treatment of fungal diseases, and more particularly to solutions of imidazole derivatives such as miconazole nitrate of sufficient strength and stability for pharmaceutical use The said composition can accommodate a therapeutically significant concentration of the antifungal agents; thereby increasing the stability of the antifungal agents in solution for extended periods of time. The solvent system comprises a primary carboxylic acid, a polar solvent, a solubilizer, a non-ionic or amphoteric surfactant, and water, in which imidazole derivatives can be dissolved.

U.S. Pat. No. 6,001,864 deals with an antifungal composition wherein an imidazole-type antifungal compound in the form of miconazole nitrate is combined with a quaternary ammonium salt. It is claimed that the miconazole nitrate is more potentiated active and has higher therapeutic effect. The composition is effective against both Trichophyton and Candida. The applicant also claims on the bases that combination disclosed in the present application has never been used before.

U.S. Pat. No. 4,911,932 relates to skin care composition which can be applied topically to prevent or treat acute inflammatory skin conditions, especially in young children. The composition is a synergistic combination of active ingredients which effectively prevent and/or treat inflammatory skin conditions such as diaper rash comprising a synergistic mixture of miconazole nitrate and zinc oxide. U.S. Pat. No. 4,911,932 claims novelty over the existing prior art over the fact that the composition in the application is more effective for the prevention and treatment of diaper rash.

U.S. Pat. No. 5,023,251 discloses a oil in water cream comprising hydrocortisone diester, oil in water emulsifier based on polyoxyethylene fatty acid esters and fatty alcohols, stearyl alcohol, white Vaseline, benzyl alcohol and water. U.S. Pat. No. 5,023,251 claims novelty on the basis that the ointments with no water or very low water are creams and are not always satisfactory in respect of absorption of the active ingredient, while the claimed invention provide an O/W cream which contains a hydrocortisone diester and which ensures satisfactory storage stability and high absorption of the active ingredient through the skin. The composition is used for the treatment of eczemas, dermatitis, psoriasis and inflammations.

U.S. Pat. No. 5,961,997 disclose antipruritic composition comprising menthol, camphor and phenol in a carrier. The composition preferably further comprises lidocaine and pramoxine and more preferably further comprise lidocaine, pramoxine and hydrocortisone acetate. The composition relieves itching in patients suffering from a variety of dermatoses or pruritis. U.S. Pat. No. 5,961,997 claims novelty on the basis that the pharmaceutical composition contains effective concentrations of relevant chemicals, while helping in avoiding components which causes allergenic, irritating, acne-causing, comedogenic, irritant dermatitis, photosensitivity, or allergic contact sensitization and yet is aesthetically pleasing. The antipruritic composition of the invention is oil-free, fragrance-free, lanolin-free and free of formaldehyde-releasing preservatives

U.S. Pat. No. 6,352,691 disclose a therapeutic after-shave care lotion comprising Aloe Vera gel, Vitamin C (Ascorbic acid), Vitamin E (tocopherol), and Hydrocortisone Acetate. U.S. Pat. No. 6,352,691 claims novelty on the assertion that the produce will provides effective relief from discomforts associated with shaving, immediate relief of irritation symptoms upon application, initiates repair of damaged skin, shall eliminate the necessity for tedious long term treatment to relieve shaving symptoms and discomforts, help in combating pseudofolliculitis, shall decrease the intensity of the natural inflammatory response caused by shaving and moisturize and nourishes the damaged skin

US 2002111298 relates to a moisturizing skin ointment composition consisting of polymyxin B Sulfate, bacitracin zinc, neomycin, hydrocortisone acetate and white petrolatum. According to US 2002111298, hydrocortisone present in the composition alleviates problems associated with itching of dry skin because the ointment penetrates the dermis almost immediately, the moisturizing properties of petrolatum allows the full benefit of the antibiotic products and hydrocortisone to remain on/in the skin through several washings thereby alleviating the need to reapply several times a day.

U.S. Pat. No. 6,767,534 deals with a post hair removal skin lotion composition for use in reducing inflammation and irritation of skin immediately following hair removal by shaving, waxing, tweezing, electrolysis, or use of depilatory products, and for repairing skin damage resulting from these methods. The composition comprises deionized water, Aloe vera gel, soybean oil, alpha lipoic acid, stearic acid, glyceryl monostearate, propylene glycol, lauramide DEA, vitamin E (tocopherol), hydrocortisone acetate, vitamin C (ascorbic acid), carbomer, hydroxymethylcellulose, methylparaben, propylparaben, and polyquaternium-15. The composition claims novelty over the existing prior art on the assumption that the current composition is more suitable for the prevention and treatment of skin damage caused by shaving and other processes used for hair removal. It also claims to provide an effective treatment for pseudofolliculitis and to prevent long-term damage to the skin.

None of the above mentioned patent applications teach or suggest:
Use of the cream base matrix as a functional element of the cream rather than a mere carrier for the main APIs
Use a known bio-polymer as a functional excipient along with anti fungal agent Miconazole Nitrate, and a steroid, Hydrocortisone Acetate
Providing far superior healing effects as micro-film forming, blood clotting, supporting epidermal growth, microbial electrostatic immobilization take effect simultaneously rather than one after the other as would be the case in conventional single-drug therapy
Improve overall medicinal properties of the cream, complimenting the API used in the cream matrix

There is therefore a need for a single-dose multiple API topical treatment that will be provided in a cream base, which cream base provides therapeutical value complementary to that provided by the main APIs and serves the purpose over and above that of being a mere carrier or delivery mechanism.

### OBJECTS AND ADVANTAGES OF THE INVENTIONS

There is therefore a need to provide a single dose multiple API Miconazole Nitrate & Hydrocortisone Acetate topical treatment formulation that will provide an effective treatment against fungal infections, skin inflammations and also help actively heal the skin rejuvenate.

Further objects of the present invention are to provide topical skin treatment formulations that:
Can deliver skin healing or regeneration beyond the activity of the main APIs—Miconazole Nitrate & Hydrocortisone Acetate such that the therapeutic outcome of the main APIs are enhanced.
Contain biologically active polymers (the so-called biopolymers) without compromising the stability of the formulations could be compromised if the right biopolymer is not selected.
Incorporate a functionally bio-active excipient polymer in cream matrix while retaining the functional stability of the APIs in a single dose format

### BRIEF DESCRIPTION OF FIGURES

FIG. 1-Non-homogeneous nature of creams containing chitosan with non-compatible excipient such as carbomer

FIG. 2-Film formation using chitosan

### SUMMARY OF THE INVENTION

The present invention is directed to a composition for treating fungal skin infections & skin inflammation, along with skin rejuvenation containing

a) a biopolymer in the form of Chitosan
b) A combination of active pharmaceutical ingredients (APIs), Miconazole Nitrate & Hydrocortisone Acetate used in treating fungal skin infections & skin inflammations,
c) A cream base containing primary and secondary emulsifiers, waxy materials, co-solvents, acids, preservatives, buffering agents, anti oxidants, chelating agents, and humectants.

### d) Water

The active ingredients, namely chitosan, a corticosteroid Hydrocortisone Acetate, and an antifungal agent Miconazole Nitrate, are incorporated in cream base for use in treating fungal skin infections and skin inflammation due to allergy & itching, & wounds on human skin involving contacting human skin with the above identified composition.

### DETAILED DESCRIPTION OF THE INVENTION

Other than in the operating examples, or where otherwise indicated, all numbers expressing quantities of ingredients are understood as being modified in all instances by the term “about”.

The present invention provides a uni-dose multi-API Miconazole Nitrate & Hydrocortisone Acetate formulation for topical skin treatment in the field of prescription medicaments. The prescription medication is distinct in its use as compared with the so-called cosmeceuticals. The cosmeceuticals are aimed towards beautification or betterment of a more-or-less intact skin or of a skin not suffering from a serious disorder. On the other hand, prescription skin formulations are aimed to provide treatment for serious skin disorders resulting from infections and wounds.

From the study of the prior art several lacking aspects of the existing topical treatment formulations in the field of prescription medications are evident. The prior art does not teach or suggest that:
Topical skin formulations can deliver skin healing or regeneration beyond the activity of the main APIs such that the therapeutic outcomes of the main APIs are enhanced.
The addition of biologically active polymers (the so-called biopolymers) is a complex process in which the stability of the formulations could be compromised if the right biopolymer is not selected.
Incorporation of a functionally bio-active excipient polymer in cream matrix while retaining the functional stability of the API in a single dose format of dermaceutical cream involves resolution of problems specific to the physical stability of cream matrix.

The active compounds Miconazole Nitrate, Hydrocortisone Acetate which may be employed in the present invention are well known in the art of treatment of fungal infections, skin inflammations respectively and a bio polymer for treating wounds and rejuvenating human skin involving contacting human skin with the above identified composition.

Examples of suitable biopolymer, which may be used, include, but are not limited to chitosan and the like.

Examples of suitable topical Corticosteroids, which may be used, include, but are not limited to, Betamethasone Valerate, Betamethasone dipropionate, Beclomethasone dipropionate, Clobetasol propionate, Clobetasone butyrate, Halobetasol propionate, Mometasone furoate, Halcinonide, Fluocinonide, Triamcinolone acetonide, Fluticasone propionate, Amcinonide, Diflorasone diacetate, Prednicarbate, Hydrocortisone acetate and the like.

Examples of suitable topical antifungal agents, which may be used, include, but are not limited to, Miconazole Nitrate, Oxiconazole, Clotrimazole, Ketoconazole, Terbinafine HCl, Ciclopirax, Tolnaftate, Nystatin and the like.

These active compounds Miconazole Nitrate & Hydrocortisone Acetate require a base component to be used in the pharmaceutical composition that uses the compounds, since the compounds cannot, by themselves, be deposited directly on to human skin due to their harshness.

The base component usually contains primary and secondary emulsifiers, waxy materials, co-solvents, acids, preservatives, buffering agents, anti oxidants, chelating agents, humectants and the like.

### Chitosan

Chitosan is a linear polysaccharide composed of randomly distributed β-(1-4)-linked D-glucosamine (deacetylated unit) and N-acetyl-D-glucosamine (acetylated unit). It is known to have a number of commercial uses in agriculture and horticulture, water treatment, chemical industry, pharmaceuticals and biomedics.

It's known properties include accelerated blood clotting. However, it is not known to a person skilled in the art that chitosan's behaviour with a pharmaceutical active ingredient such as an antibacterial or antifungal agent needs to be treated with caution.

It is known to have film forming, mucoadhesive and viscosity-increasing properties and it has been used as a binder and disintegrating agent in tablet formulations.

Chitosan generally absorbs moisture from the atmosphere/environment and the amount absorbed depends upon the initial moisture content, temperature and relative humidity of the environment.

It is regarded as a non-toxic and non-irritant material. It is biocompatible with both healthy and infected skin and has been shown to be biodegradable as it is derived from shrimps, squids and crabs.

Chitosan due to its unique physical property accelerates wound healing and wound repair. It is positively charged and soluble in acidic to neutral solution. Chitosan is bioadhesive and readily binds to negatively charged surfaces such as mucosal membranes. Chitosan enhances the transport of polar drugs across epithelial surfaces. Chitosan's properties allow it to rapidly clot blood, and it has recently gained approval in the USA for use in bandages and other hemostatic agents.

Chitosan is nonallergenic, and has natural anti-bacterial properties, further supporting its use. As a micro-film forming biomaterial, chitosan helps in reducing the width of the wound, controls the oxygen permeability at the site, absorbs wound discharge and gets degraded by tissue enzymes which are very much required for healing at a faster rate. It also reduces the itching by providing a soothing effect. It also acts like a moisturizer. It is also useful in treatment of routine minor cuts and wounds, burns, keloids, diabetic ulcers and venous ulcers.

Chitosan used in the present invention comes in various molecular weights ranging from 1 kdal to 5000 kdal.

Chitosan is discussed in the US Pharmacopoeia forum with regard to its functional excipient category. Since chitosan is basically a polymer, it is available in various grades depending upon the molecular weight. The various grades of chitosan include chitosan long chain, chitosan medium chain & chitosan short chain. The grades long, medium & short chain directly corresponds to the molecular weight of the chitosan.

Generally the long chain grade has a molecular weight in the range of 500,000-5,000,000 Da, the medium chain grade has a molecular weight in the range of 1,00,000-2,000,000 Da and the short chain grade has a molecular weight in the range of 50,000-1,000,000 Da.

The molecular weight of the chitosan plays an important role in the formulation. higher molecular weight chitosan imparts a higher viscosity to the system and lower molecular weight chitosan imparts a lower viscosity to the system.

However the medium chain grade chitosan delivered an optimum level of viscosity to the formulation. Since the dosage form is a cream, appropriate levels of viscosity is required to achieve a good spreadability over the skin.

The inventors finalized the chitosan medium chain grade for the present invention since it imparted the required rheologic properties to the cream without compromising the therapeutic activity of both the actives Miconazole Nitrate & Hydrocortisone Acetate and chitosan. The concentration of chitosan medium chain grade was carefully arrived based on several in house trials and Preclinical animal studies for efficacy.

### Topical Corticosteroids

Topical corticosteroids are a powerful tool for treating skin diseases. Corticosteroids include drugs such as Betamethasone Valerate, Betamethasone dipropionate, Beclomethasone dipropionate, Clobetasol propionate, Clobetasone butyrate, Halobetasol propionate, Mometasone furoate, Halcinonide, Fluocinonide, Triamcinolone acetonide, Fluticasone propionate, Amcinonide, Hydrocortisone acetate, Diflorasone diacetate, Prednicarbate, etc.

Topical corticosteroids are classified by their potency, ranging from weak to extremely potent. They include weak potent steroids, moderate potent steroids, potent steroids, very potent steroids and extremely potent steroids. The high potency steroids include Betamethasone Dipropionate, Betamethasone Valerate, Diflorasone Diacetate, Clobetasol Propionate, Halobetasol Propionate, Desoximetasone, Diflorasone Diacetate, Fluocinonide, Mometasone Furoate, Triamcinolone Acetonide, Fluticasone Propionate, etc. Low potency topical steroids include Desonide, Fluocinolone acetate, and Hydrocortisone acetate, etc. Topical corticosteroid is indicated for the relief of the inflammatory and pruritic manifestations of corticosteroid responsive dermatoses.

### Hydrocortisone Acetate

Hydrocortisone is a member of synthetic steroids used as anti-inflammatory and antipruritic agent. Its chemical name is Pregn-4-ene-3,20-dione, 11,17,21-trihydroxy-, (11β)-. Its molecular formula is C₂₁H₃₀O₅ and molecular weight 362.47.

Hydrocortisone Acetate is a white to off-white crystalline powder insoluble in water and slightly soluble in alcohol and in chloroform. It is a low potency corticosteroid indicated for the relief of the inflammatory and pruritic manifestations of corticosteroid-responsive dermatoses.

### Pharmacology

Hydrocortisone Acetate is a low potency synthetic corticosteroid with antiinflammatory, antipruritic, and vasoconstrictive properties. Hydrocortisone Acetate depresses formation, release, and activity of endogenous mediators of inflammation, including prostaglandins, kinins, histamine, liposomal enzymes, and complement system; modifies body's immune response. Hydrocortisone Acetate have been shown to have a wide range of inhibitory effects on multiple cell types (e.g. mast cells, eosinophils, neutrophils, macrophages and lymphocytes) and mediators (e.g. histamine, eicosanoids, leukotrienes, and cytokines) involved in inflammation and in the asthmatic response. These anti-inflammatory actions of corticosteroids may contribute to their efficacy in asthma and in skin lesions.

Mechanism Of Action: They enter cells where they combine with steroid receptors in cytoplasm and then the combination enters nucleus where it controls synthesis of protein, including enzymes that regulate vital cell activities over a wide range of metabolic functions including all aspects of inflammation formation of a protein that inhibits the enzyme phospholipase A₂ which is needed to allow the supply of arachidonic acid. Arachidonic acid is essential for the formation of inflammatory mediators They also act on cell membranes to alter ion permeability. They also modify the production of neurohormones.

### Pharmacokinetics

The extent of percutaneous absorption of topical corticosteroids is determined by many factors including the vehicle, the integrity of the epidermal barrier, and the use of occlusive dressings.

Topical corticosteroids can be absorbed from normal intact skin. Inflammation and/or other disease processes in the skin increase percutaneous absorption. Occlusive dressings substantially increase the percutaneous absorption of topical corticosteroids. Thus, occlusive dressings may be a valuable therapeutic adjunct for treatment of resistant dermatoses. Once absorbed through the skin, topical corticosteroids are handled through pharmacokinetic pathways similar to systemically administered corticosteroids. Corticosteroids are bound to plasma proteins in varying degrees. Corticosteroids are metabolized primarily in the liver and are then excreted by the kidneys. Some of the topical corticosteroids and their metabolites are also excreted into the bile.

Indications: Hydrocortisone Acetate is a low potency corticosteroid indicated for the relief of the inflammatory and pruritic manifestations of corticosteroid-responsive dermatoses.

### Topical Antifungals:

Topical anti-fungals are intended to target skin for fungal infections caused by tinea pedis, tinea cruris, tinea corporis etc. These include drugs like Miconazole Nitrate, Oxiconazole, Clotrimazole, Ketoconazole, Terbinafine HCl, Ciclopirax, Tolnaftate, Nystatin and the like.

Fungal infections are generally manifested with itching at the site. Antifungals act by altering the permeability of the fungal membrane by inhibiting the synthesis of sterols.

### Miconazole Nitrate

Miconazole Nitrate is an antifungal agent with similar antimicrobial activity to ketoconazole. Chemically, Miconazole Nitrate is 1-[2-(2,4-Dichlorophenyl)-2-[(2,4-dichlorophenyl)methoxy]ethyl]-1H-imidazole with the empirical formula C₁₈H₁₄Cl₄N₂C.HNO₃, and a molecular weight of 479.15.

Miconazole Nitrate is a White or almost white, crystalline or micro-crystalline powder, freely soluble in methanol; slightly soluble in ethanol (95%) and in chloroform; very slightly soluble in water and in ether.

It is administered by intravenous infusion in the treatment of severe systemic fungal infections including candidiasis, coccidioidomycosis, cryptococcosis, paracoccidioidomycosis, and infections due to Pseudeliescheria boydii.

Miconozole may be given by mouth for the treatment of oral and intestinal candidiasis.

It has been given prophylactically to patients at high risk of opportunistic fungal infections.

In fungal meningitis, intravenous treatment may be supplemented with intrathecal injections of Miconazole. Miconazole nitrate is used locally for treating various fungal skin infections.

### Pharmacology

Miconazole nitrate is a synthetic antifungal agent which inhibits the growth of the common dermatophytes, Trichophyton rubrum, Trichophyton mentagrophytes, and Epidermophyton floccosum, the yeast-like fungus, Candida albicans, and the organism responsible for tinea versicolor (Malassezia furfur).

Mechanism of Action: Miconazole nitrate inhibits biosynthesis of ergosterol, damaging the fungal cell wall membrane, which increases permeability causing leaking of nutrients

Pharmacokinetics: Absorption of Miconazole nitrate is negligible by topical route. Miconazole nitrate is widely distributed to body tissues; penetrates well into inflamed joints, vitreous humor of eye, and peritoneal cavity, but poorly into saliva and sputum; crosses blood-brain barrier but only to a small extent Protein binding of Miconazole nitrate is about 91% to 93%. Miconazole nitrate is metabolized in liver and is excreted in feces (˜50%) and urine (<1% as unchanged drug)

Indications: For topical application in the treatment of tinea pedis (athlete's foot), tinea cruris, and tinea corporis caused by Trichophyton rubrum, Trichophyton mentagrophytes, and Epidermophyton floccosum, in the treatment of cutaneous candidiasis (moniliasis), and in the treatment of tinea versicolor.

Most of the topical products are formulated as either creams or ointments. A cream is a topical preparation used for application on the skin. Creams are semi-solid emulsions, which are mixtures of oil and water in which APIs (Active Pharmaceutical Ingredients) are incorporated. They are divided into two types: oil-in-water (O/W) creams which compose of small droplets of oil dispersed in a continuous water phase, and water-in-oil (W/O) creams which compose of small droplets of water dispersed in a continuous oily phase. Oil-in-water creams are user-friendly and hence cosmetically acceptable as they are less greasy and more easily washed with water. An ointment is a viscous semisolid preparation containing APIs, which are used topically on a variety of body surfaces. The vehicle of an ointment is known as ointment base. The choice of a base depends upon the clinical indication of the ointment, and the different types of ointment bases normally used are:
Hydrocarbon bases, e.g. hard paraffin, soft paraffin
Absorption bases, e.g. wool fat, bees wax

Both above bases are oily and greasy in nature and this leads to the undesired effects like difficulty in applying & removal from the skin. In addition this also leads to staining of the clothes. Most of the topical products are available as cream formulation because of its cosmetic appeal.

The acidic scale of pH is from 1 to 7, and the base scale of pH is from 7 to 14. Human skins pH value is some where between 4.5 and 6. Newborn baby's skin pH is closer to neutral (pH 7), but it quickly turns acidic. Nature has designed this probably to protect young children's skin, since acidity kills bacteria. As people become older, the skin becomes more and more neutral, and won't kill as many bacteria as before. This is why the skin gets weak and starts having problems. The pH value goes beyond 6 when a person actually has a skin problem or skin disease. This shows that it is necessary to choose topicals that have a pH value close to that of skin of a young adult.

A slight shift towards the alkaline pH would provide a better environment for microorganisms to thrive. Most of the topical products are available as creams. Active compounds in cream formulations are available in ionized state, whereas in case of ointments these are present in non-ionized state. Generally, the cream formulations are the first choice of the formulators in design and development of topical dosage forms, as the cream formulations are cosmetically elegant, and also as the active compound is available in ionized state, and the drug can penetrate the skin layer fast which makes the formulation totally patient friendly.

The pH of the cream of the present invention with a functional biopolymer such as chitosan with Miconazole Nitrate & Hydrocortisone Acetate is from about 3 to 6. On the other hand, ointments that are commercially available are greasy and cosmetically non elegant. Furthermore, as the active compound in an ointment is in non-ionized form, the penetration of skin is slow.

It is essential that the active drug penetrates the skin for the optimum bio-dermal efficacy. The particle size of the active drug plays an important role here. It is necessary that the active drug is available in colloidal or molecular dispersed state for the product being highly efficacious form. Also this is to be achieved in the safe pH compatible environment of skin (4.0 to 6.0). To achieve all these, it is essential to choose proper vehicles or co-solvents for the dissolution or dispersion of the drug. The product of the present invention is highly efficacious due to the pronounced antifungal/antiinflammatory & wound healing activity of the active ingredients Miconazole Nitrate & Hydrocortisone Acetate, which are available in ultra micro-size, colloidal form, which enhances skin penetration.

### Rationale For Miconazole Nitrate, Hydrocortisone Acetate And Chitosan Combination:

Numerous topical treatments are currently employed for the treatment of fungal infections and skin inflammations. However there is no effective single-dose therapy for protecting the skin, controlling superficial bleeding, wounds and burns. To meet this need and to bring affordable and safe therapy to the dispersed segment of population across all countries/communities, a therapy with unique combination of chitosan, a biopolymer with skin rejuvenation properties with Miconazole Nitrate & Hydrocortisone Acetate is proposed as a novel cream.

Topical Miconazole Nitrate have profound efficacy in skin infections of varied etiology due to its antifungal properties. A drawback of the monotherapy with topical antifungal like Miconazole Nitrate has been the relatively slow onset of the effect.

Steroids like Hydrocortisone Acetate provide much wanted rapid relief of the pruritus. Combining Miconazole Nitrate with topical Hydrocortisone Acetate is expected to provide fast relief because of the steroid effect and a lingering post treatment antifungal effect allowing for an overall reduction in intermittent use of the product. Generally topical steroids of high potency are used for duration of one to two weeks; for low potency steroids the period may be three to four weeks.

By employing Hydrocortisone Acetate, Miconazole Nitrate & chitosan in a formulation, the properties of steroids, antifungals and chitosan are optimized. As chitosan is film forming, biocompatible, non-allergenic material it helps in protecting the skin by acting as a barrier. It further controls the superficial bleeding caused by scratching and also arrests the mobility of pathogens due to its cationic charge.

The properties of Hydrocortisone Acetate, Miconazole Nitrate and chitosan's skin regenerative aspects are well exploited in the present invention and the maximum therapeutic benefit is passed on to the patient thereby aiding in faster healing. This ensures that the patient would benefit for the treatment of skin dermatitis, eczema, wounds, and fungal infections.

The inclusion of chitosan in the formulation takes care of many attributes, which are considered to be very much essential in treating skin ailments. The combination of chitosan with Hydrocortisone Acetate and Miconazole Nitrate is unique and novel since this is not available commercially across the globe.

The concept of the combination is justified by considering the physical, chemical and therapeutic properties of chitosan used in combination with Hydrocortisone Acetate, Miconazole Nitrate.

### Inventive Aspects of the Present Invention:

Another inventive aspect of the present invention is that the addition of a functional excipient in the cream base is not a straight forward process of mere addition. The inventor has found that the compatibility of the functional excipient such as chitosan with other agents in the cream is of critical importance. This is because incompatibility would compromise the stability of the final product. As examples, the inventors have found that well known excipients such as Xanthan Gum and carbomer which have been variously used as stabilising agents, cannot be used in combination with functional biopolymers such as chitosan.

Excipients for topical dosage forms include Polymers, Surfactants, Waxy Materials, and Emulsifiers etc. Polymers are used as gelling agents, suspending agents, viscosity builders, release modifiers, diluents, etc. Surfactants are used as wetting agents, emulsifiers, solubilising agents release enhancers, etc.

Generally Polymers & Surfactants may or may not possess ionic charge. They may be anionic or cationic or non-ionic in nature. If anionic excipients are included in the formulation they interact with cationic formulation excipients and produce products which are not homogenous, aesthetically not appealing and give rise to unwanted by products, possible allergens, impurities, toxic substances etc due to incompatibility.

Since the dosage is for the treatment of ailing patients, these incompatibilities in the products cannot be accepted and these add more complication to the patients.

The inventors carefully screened the excipients which included the Polymers and Surfactants for developing a formulation. A thorough study was performed after screening the short listed excipients. The possible interactions between the excipients were given much focus and detailed experiments were done.

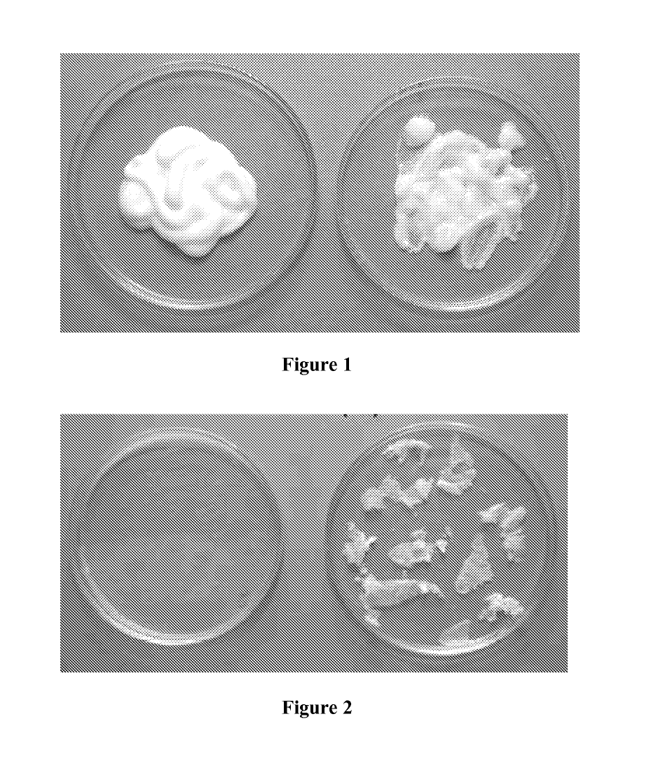

To quote some examples about the anionic-cationic interaction in the cream dosage form the inventors made some formulations of Hydrocortisone Acetate and Miconazole Nitrate (see tables 1-5) containing Xanthan Gum & Chitosan, Acrylic acid polymer & Chitosan, Sodium Lauryl Sulphate & Chitosan, Docusate Sodium & Chitosan and Gum Arabic & Chitosan. The results clearly indicated the occurrence of interactions which was very much visible and seen as lumps into the entire system. The final product was also not aesthetically appealing without homogeneity. The attached FIG. 1 clearly explains the interaction between chitosan and unsuitable anionic excipients. Based on the observations and thorough knowledge about the excipients, the inventors arrived at a robust formula without any possible interactions.

TABLE 1
Formulation of Miconazole Nitrate & Hydrocortisone
Acetate Cream with Chitosan and Xanthan Gum

| S.No. | Ingredients | % W/W |
|---|---|---|
| 1 | Hydrocortisone Acetate | 1.0 |
| 2 | Miconazole Nitrate | 2.0 |
| 3 | Chitosan | 0.25 |
| 4 | Lactic acid | 0.1 |
| 5 | Xanthan Gum | 1.0 |
| 6 | Cetostearyl Alcohol | 8.5 |
| 7 | White Soft Paraffin | 8.5 |
| 8 | Polyoxyl 20cetostearyl ether (Cetomacrogol 1000) | 2.5 |
| 9 | Light Liquid Paraffin | 5 |
| 10 | Propylene Glycol | 15 |
| 11 | Methyl Paraben | 0.2 |
| 12 | Propyl Paraben | 0.02 |
| 13 | Disodium Edetate | 0.1 |
| 14 | Disodium hydrogen Orthophosphate anhydrous | 0.5 |
| 15 | Purified Water | 55.5 |

TABLE 2
Formulation of Miconazole Nitrate & Hydrocortisone Acetate
Cream with Chitosan and Acrylic Acid Polymer

| S.No. | Ingredients | % W/W |
|---|---|---|
| 1 | Hydrocortisone Acetate | 1.0 |
| 2 | Miconazole Nitrate | 2.0 |
| 3 | Chitosan | 0.25 |
| 4 | Lactic acid | 0.1 |
| 5 | Acrylic Acid Polymer | 0.75 |
| 6 | Cetostearyl Alcohol | 8.5 |
| 7 | White Soft Paraffin | 8.5 |
| 8 | Polyoxyl 20cetostearyl ether (Cetomacrogol 1000) | 2.5 |
| 9 | Light Liquid Paraffin | 5 |
| 10 | Propylene Glycol | 15 |
| 11 | Methyl Paraben | 0.2 |
| 12 | Propyl Paraben | 0.02 |
| 13 | Disodium Edetate | 0.1 |
| 14 | Disodium hydrogen Orthophosphate anhydrous | 0.5 |
| 15 | Purified Water | 55.5 |

TABLE 3
Formulation of Miconazole Nitrate & Hydrocortisone Acetate
Cream with Chitosan and Sodium Lauryl Sulphate

| S.No. | Ingredients | % W/W |
|---|---|---|
| 1 | Hydrocortisone Acetate | 1.0 |
| 2 | Miconazole Nitrate | 2.0 |
| 3 | Chitosan | 0.25 |
| 4 | Lactic acid | 0.1 |
| 5 | Sodium Lauryl Sulphate | 1.0 |
| 6 | Cetostearyl Alcohol | 8.5 |
| 7 | White Soft Paraffin | 8.5 |
| 8 | Polyoxyl 20cetostearyl ether (Cetomacrogol 1000) | 2.5 |
| 9 | Light Liquid Paraffin | 5 |
| 10 | Propylene Glycol | 15 |
| 11 | Methyl Paraben | 0.2 |
| 12 | Propyl Paraben | 0.02 |
| 13 | Disodium Edetate | 0.1 |
| 14 | Disodium hydrogen Orthophosphate anhydrous | 0.5 |
| 15 | Purified Water | 55.5 |

TABLE 4
Formulation of Miconazole Nitrate & Hydrocortisone
Acetate Cream with Chitosan and Docusate Sodium

| S.No. | Ingredients | % W/W |
|---|---|---|
| 1 | Hydrocortisone Acetate | 1.0 |
| 2 | Miconazole Nitrate | 2.0 |
| 3 | Chitosan | 0.25 |
| 4 | Lactic acid | 0.1 |
| 5 | Docusate Sodium | 1.0 |
| 6 | Cetostearyl Alcohol | 8.5 |
| 7 | White Soft Paraffin | 8.5 |
| 8 | Polyoxyl 20cetostearyl ether (Cetomacrogol 1000) | 2.5 |
| 9 | Light Liquid Paraffin | 5 |
| 10 | Propylene Glycol | 15 |
| 11 | Methyl Paraben | 0.2 |
| 12 | Propyl Paraben | 0.02 |
| 13 | Disodium Edetate | 0.1 |
| 14 | Disodium hydrogen Orthophosphate anhydrous | 0.5 |
| 15 | Purified Water | 55.5 |

TABLE 5
Formulation of Miconazole Nitrate & Hydrocortisone
Acetate Cream with Chitosan and Gum Arabic

| S.No. | Ingredients | % W/W |
|---|---|---|
| 1 | Hydrocortisone Acetate | 1.0 |
| 2 | Miconazole Nitrate | 2.0 |
| 3 | Chitosan | 0.25 |
| 4 | Lactic acid | 0.1 |
| 5 | Gum Arabic | 1.0 |
| 6 | Cetostearyl Alcohol | 8.5 |
| 7 | White Soft Paraffin | 8.5 |
| 8 | Polyoxyl 20cetostearyl ether (Cetomacrogol 1000) | 2.5 |
| 9 | Light Liquid Paraffin | 5 |
| 10 | Propylene Glycol | 15 |
| 11 | Methyl Paraben | 0.2 |
| 12 | Propyl Paraben | 0.02 |
| 13 | Disodium Edetate | 0.1 |
| 14 | Disodium hydrogen Orthophosphate anhydrous | 0.5 |
| 15 | Purified Water | 55.5 |

The above products (tables 1 to 5) are examples of products that do not form homogeneous creams, and produce non-homogeneous creams of the type illustrated in FIG. 1. Yet the proportions stated in these examples are some things that a person skilled in the art may use based on currently available knowledge. Only after a thorough and extensive trials and errors would it be possible to arrive at right types and proportions of excipients.

As we have discussed earlier, in a combination therapy, steroids like Hydrocortisone Acetate provide relief against inflammation and antifungals like Miconazole Nitrate provide relief against fungal infections. However, the aspects such as like skin protection, bleeding at the site, mobility of pathogens from one site to another, etc are not addressed so far in a single dose therapy.

This present invention with its single-dose application fills this gap by incorporating chitosan and tapping the required benefits of skin protection (by way of film forming property), stopping the bleeding (by way of blood clotting property) and immobilization of pathogenic microbes (due to its cationic electrostatic property).

Therapeutic value addition by incorporation of a functional excipient in the form of a chitosan which is a biopolymer in the cream matrix. The value addition is an integrated sub-set of the following functional attributes of the biopolymer:
formulation of a micro-film on the skin surface
accelerated blood clotting as compared to creams that do not contain film-forming biopolymers
electrostatic immobilisation of surface microbes due to cationic charge of the biopolymer
significant enhancement of the skin epithelisation or regeneration

The inventive efforts involved in developing the platform technology covered by incorporation of a functional biopolymer in prescription dermaceutical products are:
in identification of the complementary therapeutic value that such incorporation delivers
in identification of issues related to physio-chemical stability of the product resulting from the incorporation of the biopolymer
in providing a single dose format where the fungal infection and inflammation has been identified

The importance of a single dose treatment, particularly in the underdeveloped countries cannot be overemphasized. In absence of access to a general physician in most parts of south Asia or Africa, let alone a skin specialist, a single dose formulation dramatically increases chances of eliminating root cause of the skin disorder while also allowing the skin to regenerate.

During dermatological conditions, currently available therapies do not address the issues like protecting the skin, arresting the bleeding etc. The unique innovative formulation of the present invention takes care of the skin conditions by treating them along with controlling the superficial bleeding at the site. It is well understood that if the superficial bleeding is left untreated, it will lead to secondary microbial infections. The present invention advantageously provides a solution to this unmet need.

Further, with ever increasing pressures on medical support systems and the attendant scarcity/high cost of the same, there is an emergent need all across the globe to address the following issues in such cases—
Patients waiting too long for treatment
Staying unnecessarily long when they get to hospital
Having to come back more often than they need to

Reducing the length of stay is a key underlying problem to be tackled in most cases. The present invention with its single-dose therapy reduces the overall treatment time of a serious skin disorder significantly.

### Preferred Embodiment 1

A novel dermaceutical cream for topical treatment of fungal skin infections, inflammations, and for related wound healing, wherein said cream comprises an antifungal agent, Miconazole Nitrate, a corticosteroid, Hydrocortisone Acetate and a biopolymer provided in a cream base, said cream base comprising at least one of each of a preservative, a primary and a secondary emulsifier, a waxy material, a co-solvent, an acid, and water, preferably purified water.

### Embodiment No. 1

A novel dermaceutical cream as disclosed in the preferred embodiment no. 1, wherein said cream further comprising any of a group comprising a buffering agent, an antioxidant, a chelating agent, a humectant, or any combination thereof.

### Embodiment No. 2

A novel dermaceutical cream as disclosed in the preferred embodiment no. 1 wherein
said antifungal agent Miconazole Nitrate is added in an amount between about 0.05% w/w and about 5% w/w, preferably between 0.1 and 3.0% w/w, more preferably 2.0 w/w; and
said corticosteroid Hydrocortisone Acetate is added in an amount between about 0.001% (w/w) and about 5% (w/w), preferably between about 0.01% and about 2.5% w/w, more preferably 1 w/w and,
said biopolymer is in the form of chitosan, added in an amount between about 0.01% and about 1% by weight, preferably added in an amount from about 0.01% w/w to about 0.5% w/w and most preferably about 0.25% w/w, said chitosan being US Pharmacopoeia conformant with regard to its functional excipient category and selected from any grades such as Long Chain, Medium Chain & Short Chain, and has a molecular weight in the range between 50 kDa to 5000 kDa,

said primary and secondary emulsifiers are selected from a group comprising Cetostearyl alcohol, Cetomacrogol-1000, Polysorbate-80, Span-80, and the like and added in an amount from about 1% (w/w) to 20% (w/w); said waxy materials is selected from a group comprising white soft paraffin, liquid paraffin, hard paraffin and the like, or any combination thereof, and added in an amount from about 5% (w/w) to 30% (w/w); said co-solvent is selected from a group comprising Propylene Glycol, Hexylene Glycol, PolyEthylene Glycol-400, Isopropyl Myristate and the like, or any combination thereof, and added in an amount from about 5% (w/w) to 50% (w/w); said acid is selected from a group comprising HCl, H₂SO₄, HNO₃, Lactic acid and the like, or any combination thereof, and added in an amount from about 0.005% (w/w) to 0.5% (w/w); said preservative is selected from a group comprising Methylparaben, Propylparaben, Chlorocresol, Potassium sorbate, Benzoic acid and the like, or any combination thereof, and added in an amount from about 0.05% (w/w) to 0.5% (w/w); said water is added in the amount in the range of 20% (w/w) to 75% (w/w), preferably 45% (w/w) to 65% (w/w), more preferably 55% (w/w) to 62% (w/w), preferably purified water.

### Embodiment No. 3

A novel cream as disclosed in the preferred embodiment no. 1 and the embodiment no. 2, further comprising a buffering agent which is selected from a group comprising Di Sodium Hydrogen Ortho Phosphate, Sodium Hydrogen Ortho Phosphate and the like, or any combination thereof, and added in an amount from about 0.05% (w/w) to 1.00% (w/w).

### Embodiment No. 4

A novel cream as disclosed in the preferred embodiment no. 1 and the embodiments no. 2 and 3, further comprising an antioxidant which is selected from a group comprising Butylated Hydroxy Anisole, Butylated Hydroxy Toluene and the like, or any combination thereof, and added in an amount from about 0.05% (w/w) to 5% (w/w).

### Embodiment No. 5

A novel cream as disclosed in the preferred embodiment no. 1 and the embodiments no. 2 to 4, further comprising a chelating agent which is selected from a group comprising Disodium EDTA and the like, or any combination thereof, and added in an amount from about 0.05% (w/w) to 1% (w/w).

### Embodiment No. 6

A novel cream as disclosed in the preferred embodiment no. 1 and the embodiments no. 2 to 4, further comprising a humectant which is selected from a group comprising Glycerin, Sorbitol, Propylene Glycol and the like, or any combination thereof, and added in an amount from about 5% (w/w) to 50% (w/w).

### Embodiment No. 7

A process of making a cream is disclosed, said process comprising the steps of providing an antifungal agent, Miconazole Nitrate, a corticosteroid, Hydrocortisone Acetate, and a biopolymer in a cream base comprising at least one of each of a preservative, a primary and a secondary emulsifier, a waxy material, a co-solvent, an acid, and water, preferably purified water, and mixing all the ingredients together to form a homogeneous cream.

### Embodiment No. 8

A process of making a cream as disclosed in the embodiment no. 7, wherein the ingredients further comprise any of a group comprising a buffering agent, an antioxidant, a chelating agent, a humectant, or any combination thereof.

### Embodiment No. 9

A novel cream as disclosed in any of the foregoing embodiments, wherein chitosan has a molecular weight range of 1 kdal to 5000 kdal.

The present invention will be further elucidated with reference to the accompanying examples containing the composition and stability studies data, which are however not intended to limit the invention in any way whatever.

### Example-I:

TABLE 6
Miconazole Nitrate (2%) + Hydrocortisone
Acetate (1%) + Chitosan Cream

| S.No. | Ingredients | % W/W |
|---|---|---|
| 1 | Hydrocortisone Acetate | 1.0 |
| 2 | Miconazole Nitrate | 2.0 |
| 3 | Chitosan | 0.25 |
| 4 | Lactic acid | 0.1 |
| 5 | Cetostearyl Alcohol | 8.5 |
| 6 | White Soft Paraffin | 8.5 |
| 7 | Polyoxyl 20cetostearyl ether (Cetomacrogol 1000) | 2.5 |
| 8 | Light Liquid Paraffin | 5 |
| 9 | Propylene Glycol | 15 |
| 10 | Methyl Paraben | 0.2 |
| 11 | Propyl Paraben | 0.02 |
| 12 | Disodium Edetate | 0.1 |
| 13 | Disodium hydrogen Orthophosphate anhydrous | 0.5 |
| 14 | Purified Water | 56.5 |

A comparison of table 6, and tables 1 to 5 will illustrate the difference in the products that would be based on the conventional drug design and the innovative approach adopted in the present invention.

APIs-stability experiments were carried out (see tables 7-9) using the product of the present invention. Tests were carried out to observe (or measure as appropriate) the physical appearance of the product, the pH value and assay of the APIs over a period of time. The product used for the Stability Studies tests contained approximately 10% extra APIs (overages). It was packaged in an aluminium collapsible tube. Detailed test results for the product have been presented. The % of the corticosteroid, Hydrocortisone Acetate and the antifungal, Miconazole Nitrate used in all examples are measured w/w with respect to the final product.

### Product: Hydrocortisone Acetate+Miconazole Nitrate Cream

PACK: Aluminum Collapsible tube
Composition: Hydrocortisone Acetate IP 1.0% w/w, Miconazole Nitrate IP 2.0% w/w

TABLE 7
Description Test, Batch No. HAM-03

| | | 1st | 2nd | 3rd |
|---|---|---|---|---|
| Conditions | Initial | Month | Month | Month |
| 40° C./75% RH | Homogenous White | same as | same as | same as |
| | to off White viscous | initial | initial | initial |
| | cream | | | |
| 30° C./65% RH | | Do | Do | Do |
| 25° C./60% RH | | Do | Do | Do |
| Temp cycling | | Do | — | — |
| Freezthaw | | Do | — | — |

Measured parameter: Physical appearance
Best value of measured parameter: Homogeneous White to off White Viscous cream;
Method of measurement: Observation by naked eye

TABLE 8
Assay (%) Test, Batch No. HAM-03

| | | | 1st | 2nd | 3rd |
|---|---|---|---|---|---|
| Conditions | Assay (%) | Initial | Month | Month | Month |
| 40° C./75% RH | i) Hydrocorti- | 108.68 | 108.66 | 108.56 | 108.36 |
| | sone Acetate | | | | |
| | ii) Miconazole | 108.62 | 108.55 | 108.52 | 108.40 |
| | Nitrate | | | | |
| 30° C./65% RH | i) Hydrocorti- | — | 108.65 | 108.62 | 108.46 |
| | sone Acetate | | | | |
| | ii) Miconazole | | 108.58 | 108.41 | 108.25 |
| | Nitrate | | | | |
| 25° C./60% RH | i) Hydrocorti- | — | 108.62 | 108.52 | 108.38 |
| | sone Acetate | | | | |
| | ii) Miconazole | | 108.64 | 108.40 | 108.31 |
| | Nitrate | | | | |
| Temp cycling | i) Hydrocorti- | — | 108.15 | — | — |
| | sone Acetate | | | | |
| | ii) Miconazole | | 108.11 | — | — |
| | Nitrate | | | | |
| Freezthaw | i) Hydrocorti- | — | 108.25 | — | — |
| | sone Acetate | | | | |
| | ii) Miconazole | — | 108.25 | — | — |
| | Nitrate | | | | |

Measured parameter: Assay (%);
Limits of measured parameter: 90-110
Method of measurement: HPLC Method

TABLE 9
pH Test, Batch No. HAM-03

| | | 1st | 2nd | 3rd |
|---|---|---|---|---|
| Conditions | Initial | Month | Month | Month |
| 40° C./75% RH | 4.55 | 4.54 | 4.54 | 4.53 |
| 30° C./65% RH | — | 4.54 | 4.54 | 4.53 |
| 25° C./60% RH | — | 4.55 | 4.54 | 4.54 |
| Temperature cycling | — | 4.51 | — | — |
| Freezthaw | — | 4.52 | — | — |

Measured parameter: pH;
Limits of measured parameter: 3-6
Method of measurement: Digital pH Meter

### Method of Application of the Cream:

The cream is applied after thorough cleansing and drying the affected area. Sufficient cream should be applied to cover the affected skin and surrounding area. The cream should be applied two-four times a day depending upon the skin conditions for the full treatment period, even though symptoms may have improved.

### Experiments:

Experiments were carried out with the cream in laboratory as well as using suitable animal models inflicted with excision wounds. Four aspects were tested—wound contraction, epithelisation, blood clotting time, and film forming These aspects together would suggest that the microbes were immobilized thereby leading to effective wound healing.

### A. Wound Contraction:

Excision wound healing activity of the cream of the present invention was determined through animal testing. An excision wound 2.5 cm in diameter was inflicted by cutting away full thickness of the skin. The amount of contraction of the wound observed over a period indicated that the cream of present invention provides significantly improved wound contraction than that achieved through application of a conventional cream.

### B. Period of Epithelisation:

Epithelisation of the wound occurred within shorter number of days using the cream of the present invention as compared to the days taken for epithelisation using the conventional cream Therefore one benefit of the cream of the present invention is that it facilitates faster epithelisation of the skin than through the use of conventional creams.

### C. Blood Clotting:

Blood clotting time was observed in both groups of animals, untreated control group and the test group of animals treated with the product of the present invention. Statistically significant decrease in the blood clotting time in treated group animals was observed when compared with that of the control group animals. The mean percent reduction of 25-65% was observed for the blood clotting time using the product of the present invention.

### Film Forming Properties:

It is evident from FIG. 1 that chitosan does not lose its film forming property in the presence of the excipients used for cream preparations in the present invention.

### Results And Discussion:

It is evident that the properties of chitosan when used in formulations containing the excipients used in the current invention are not compromised in any way. This has been achieved through a careful selection of excipients. For example, our experiments show that widely used excipients such as xanthan gum or carbomer precipitate in combination with chitosan due to cationic, anionic interactions.

The therapeutic impact, as observed from the animal testing, of the addition of chitosan to corticosteroid, Hydrocortisone Acetate & antifungal agent, Miconazole Nitrate is shown in the following table by considering various aspects of therapeutic cure of a compromised skin condition:

TABLE 10

| | Existing | Products of the present |
|---|---|---|
| Therapeutic aspect | creams | invention |
| 1. Blood Clotting | None | Statistically significant reduction |
| time | explicitly | in clotting time as evidenced by |
| | claimed | pre-clinical animal trials |
| 2. Immobilisation | None | Expected to immobilise the |
| of microbes | explicitly | surface microbes because of the |
| | claimed | cationic charge of chitosan |
| 3. Epidermal | None | It is well known that chitosan |
| growth support | explicitly | possesses properties that have |
| | claimed | significant complimentary action |
| | | on epidermal growth. This |
| | | functional aspect of chitosan is |
| | | preserved in the product of the |
| | | present invention |
| 4. Micro-film | None | Yes (see FIG. 2) |
| forming | explicitly | |
| | claimed | |
| 5. Overall wound | Standard as | Provides superior healing |
| healing medicinal | per existing | properties |
| effect | products | |

It is evident that the film forming ability of the chitosan incorporated in the cream allows better access of the antifungal agent, Miconazole Nitrate and the corticosteroid, Hydrocortisone Acetate to the infected/inflamed area and results in better functioning of these APIs.

The therapeutic efficacy of topically applied cream of the present invention is due to the pronounced antifungal activity of the Miconazole Nitrate against the organisms responsible for skin infections, the antiallergic & anti-inflammatory property of corticosteroid, Hydrocortisone Acetate, the unique ability of actives to penetrate intact skin and wound healing & soothing properties of chitosan.

It is evident from the foregoing discussion that the present invention offers the following advantages and unique aspects over the currently available dermaceutical compositions for fungal infections & inflammations:
1. The cream of the present invention incorporates a skin-friendly biopolymer in the form of chitosan provides enhanced therapeutic outcomes. This is evident from the reduced blood clotting time, increased epithelial effect, and faster relief from infection and inflammation.
2. The cream of the present invention incorporates a biopolymer without compromising the stability of the cream matrix and without adversely affecting the functioning of known active pharmaceutical ingredients. This has been achieved through a careful selection of functional excipients to bypass undesirable aspects of physio-chemical compatibility/stability and bio-release.
3. The cream of the present invention provides an integrated uni-dose or a single-dose therapy hitherto unavailable in prescription dermaceutical formulations.
4. The novel cream of the present invention is adequately stable/efficacious at ambient conditions and does not need special temperature control during transportation/storage—hence will go a long way in achieving these social objectives.

According to another embodiment of the present invention, there is also provided a process for treating fungal skin infections, skin inflammations, and wound healing involving contacting human skin with the above-disclosed composition.

While the above description contains much specificity, these should not be construed as limitation in the scope of the invention, but rather as an exemplification of the preferred embodiments thereof. It must be realized that modifications and variations are possible based on the disclosure given above without departing from the spirit and scope of the invention. Accordingly, the scope of the invention should be determined not by the embodiments illustrated, but by the appended claims and their legal equivalents.

1. A medicinal cream for topical treatment of fungal skin infections, inflammations, and for related wound healing, wherein said cream comprises an antifungal agent in the form of Miconazole Nitrate, a corticosteroid in the form of Hydrocortisone Acetate, and a biopolymer provided in a cream base, said cream base comprising at least one of each of a preservative, a primary and a secondary emulsifier, a waxy material, a co-solvent, an acid, and water, preferably purified water, said biopolymer being preferably chitosan.

2. A medicinal cream as claimed in claim 1, wherein said cream further comprising any of a group comprising a buffering agent, an antioxidant, a chelating agent, a humectant, or any combination thereof.

3. A medicinal cream as disclosed in claim 2 wherein:
said antifungal agent Miconazole Nitrate is added in an amount between about 0.05% w/w and about 5% w/w, preferably between 0.1 and 3.0% w/w; and more preferably about 2.0% w/w and
said corticosteroid Hydrocortisone Acetate is added in an amount between about 0.001% (w/w) and about 5% (w/w), preferably between about 0.01% w/w and about 2.5% w/w, and more preferably about 1% w/w, and
said biopolymer is in the form of chitosan, added in an amount between about 0.01% and about 1% by weight, preferably from about 0.01% w/w to about 0.5% w/w and most preferably about 0.25% w/w,
said primary and secondary emulsifiers are selected from a group comprising Cetostearyl alcohol, Cetomacrogol-1000, Polysorbate-80, Span-80 and the like and added in an amount from about 1% (w/w) to 20% (w/w); said waxy materials is selected from a group comprising white soft paraffin, liquid paraffin, hard paraffin and the like, or any combination thereof, and added in an amount from about 5% (w/w) to 30% (w/w); said co-solvent is selected from a group comprising Propylene Glycol, Hexylene Glycol, PolyEthylene Glycol-400, Isopropyl Myristate and the like, or any combination thereof, and added in an amount from about 5% (w/w) to 50% (w/w); said acid is selected from a group comprising HCl, H₂SO₄, HiNO₃, Lactic acid and the like, or any combination thereof, and added in an amount from about 0.005% (w/w) to 0.5% (w/w); said preservative is selected from a group comprising Methylparaben, Propylparaben, Chlorocresol, Potassium sorbate, Benzoic acid and the like, or any combination thereof, and added in an amount from about 0.05% (w/w) to 0.5% (w/w); said water is added in the amount in the range of 20% (W/w) to 75% (w/w), preferably 45% (w/w) to 65% (w/w), more preferably 55% (w/w) to 62% (w/w), preferably purified water.

4. A medicinal cream as claimed in claim 3 further comprising a buffering agent which is selected from a group comprising Di Sodium Hydrogen Ortho Phosphate, Sodium Hydrogen Ortho Phosphate and the like, or any combination thereof, and added in an amount from about 0.05% (w/w) to 1.00% (w/w).

5. A medicinal cream as claimed in claim 4 further comprising an antioxidant which is selected from a group comprising Butylated Hydroxy Anisole, Butylated Hydroxy Toluene and the like, or any combination thereof, and added in an amount from about 0.001% (w/w) to 1% (w/w).

6. A medicinal cream as claimed in claim 5 further comprising a chelating agent which is selected from a group comprising Disodium EDTA and the like, or any combination thereof, and added in an amount from about 0.05% (w/w) to 1% (w/w).

7. A medicinal cream as claimed in claim 6 further comprising a humectant which is selected from a group comprising Glycerin, Sorbitol, Propylene Glycol and the like, or any combination thereof, and added in an amount from about 5% (w/w) to 50% (w/w).

8. A process of making a cream, said process comprising the steps of providing an antifungal agent—Miconazole Nitrate, a corticosteroid—Hydrocortisone Acetate, and a biopolymer in a cream base comprising at least one of each of a preservative, a primary and a secondary emulsifier, a waxy material, a co-solvent, an acid, and water, preferably purified water, and mixing all the ingredients together to, form a homogeneous cream.

9. A process of making a cream as claimed in claim 8, wherein the ingredients further comprise any of a group comprising a buffering agent, an antioxidant, a chelating agent, a humectant, or any combination thereof.
